(19) United States
(12) Patent Application Publication
(10) Pub. No.: US 2012/0230935 A1
(43) Pub. Date: Sep. 13, 2012

(54) SOLVENT COMPOSITION FOR ONE-STEP PERMANENT WAVE AND HAIR STRAIGHTENER

(75) Inventors: Hacksoo Kim, Gyeonggi-do (KR); Bohyeon Kim, Incheon (KR); Jindong Jang, Incheon (KR); Jaehoon Hwang, Incheon (KR); Kyounghee Jang, Seoul (KR); Gijung Joe, Incheon (KR); Hyojin Moon, Gyeonggi-do (KR)

(73) Assignee: SOMANG COSMETICS CO., LTD., Incheon-city (KR)

(21) Appl. No.: 13/076,136

(22) Filed: Mar. 30, 2011

(30) Foreign Application Priority Data
Mar. 8, 2011 (KR) 10-2011-0020296

(51) Int. Cl. A61K 8/64; A61Q 5/04

(52) U.S. Cl. 424/70.51; 424/70.2; 424/70.5

(57) ABSTRACT

Disclosed herein is a solvent composition for one-step permanent wave and hair straightener, comprising: amino acid or a derivative thereof; protein or a derivative thereof; a reducing agent; an alkaline agent; an amino acid-based surfactant; organic acid; alcohol; and urea. The solvent composition does not damage hair nor irritate the scalp, does not emit an odor, and can greatly improve the efficiency of curling and straightening hair and the ability to maintain the curled and straightened hair.

### CROSS-REFERENCE TO RELATED APPLICATIONS

This application claims priority to Korean Application 10-2011-0020296, filed Mar. 8, 2011, entitled Solvent Composition for One-step Permanent Wave and Hair Straightener, which is hereby incorporated by reference in its entirety.

### TECHNICAL FIELD

Embodiments of the present invention relate to a novel solvent composition for one-step permanent wave and hair straightener, and more particularly to a novel solvent composition for one-step permanent wave and hair straightener which does not damage hair nor irritate the scalp, which does not emit an odor and which can greatly improve the efficiency of curling and straightening the hair and the ability to maintain the curled and straightened hair.

### BACKGROUND ART

Generally, the principle behind permanent wave and hair straightening is closely related to the structure and properties of hair. Hair has a three-layer structure including an epidermis layer as the outermost layer, a cortex layer as the middle layer and a medulla layer as the innermost layer. The cortex layer, which is a main component of hair, is made of highly-elastic fibrous scleroprotein, called keratin. Keratin is a kind of protein composed of polypeptides connected by the peptide bonds of various amino acids, and side-chain polymer materials are disposed between the polypeptides by cystine bonds, salt bonds, hydrogen bonds, etc. Keratin contains a large amount of sulfur compared to other proteins.

Since hair is made of keratin with such a structure, hair becomes elastic so that it returns to its original state after having been bent. Therefore, the principle behind a permanent wave is that a desired hair wave is formed by cutting the side chains of keratin to temporarily remove the elasticity of hair and then recombining the cut side chains.

In line with this principle, the composition most widely used in a permanent wave and hair straightener includes a first agent and a second agent. The first agent, which is a reducing agent including thioglycolic acid or a salt and ester thereof and cysteine or a salt or acetyl derivative thereof, reduces the cysteine bonds in hair. The second agent, which is an oxidizing agent including hydrogen peroxide or sodium borate, recombines the side chains cut by the reduction action of the first agent to wave and straighten hair. In this case, in order to improve effect and usability, an alkaline agent, a penetrating agent, a stabilizing agent, a wetting agent, a hair growing agent, a coloring agent, an emulsifying agent, a perfume, etc. may be used with the first and second agents.

The first agent is divided as follows according to the main component and use thereof: a cold two-step permanent wave agent including thioglycolic acid or a salt thereof as a main component; a cold two-step permanent wave agent including cysteine, a cysteine salt or acetyl cysteine as a main component; a hot two-step permanent wave agent including thioglycolic acid or a salt thereof as a main component; a cold two-step hair straightening agent including thioglycolic acid or a salt thereof as a main component; a cold one-step permanent wave agent including thioglycolic acid or a salt thereof as a main component; a hot two-step hair straightening agent including thioglycolic acid or a salt thereof as a main component; a hot two-step hair straightening agent including thioglycolic acid or a salt thereof as a main component and used with a hot-air dryer for high temperature styling; and an exothermic two-step permanent wave agent including thioglycolic acid or a salt thereof as a main component. Further, the first agent is prepared in the form of a liquid, lotion or cream, and is generally opaque.

Such permanent wave agents and hair straightening agents are problematic in that the bonding of keratin in hair, caused by a reducing agent and an oxidizing agent, is conducted under severe conditions, such as cutting by reduction, bonding by an oxidizing agent, high-temperature heating with a strong alkali, double exposure to a reducing agent and an oxidizing agent, and the like, so that the strength of hair becomes low, thereby greatly damaging hair.

Further, such permanent wave agents and hair straightening agents are problematic in that they must be applied two times for a long period of time, in that they irritate scalp because they have a pH in the alkali region, and in that they emit a bad odor because a reducing agent and reductive materials remain behind after they are applied.

In order to solve the above problems, a one-step permanent solvent has been developed and used. However, this one-step permanent solvent is also problematic in that it takes more time to curl hair, thus decreasing the efficiency of curling hair, and in that hair easily loses its curls.

### SUMMARY OF THE EMBODIMENTS

It is an object of the present invention is to provide a novel solvent composition for one-step permanent wave and hair straightener, which can greatly improve the efficiency of curling and straightening hair and the ability to maintain the curled and straightened hair. The present inventors found that the efficiency of curling and straightening hair and the ability to maintain the curled and straightened hair can be improved when a solvent composition for one-step permanent wave and hair straightener includes: a specific amino acid-based surfactant which has high affinity for hair components (protein and amino acid), which induces a rapid cystine reduction reaction, and which can improve the penetration and action of a reducing agent by decreasing the repellency between the amino acid-based surfactant and hair protein; organic acid accelerating the cleaving and natural oxidizing of cystine bonds; and urea accelerating the absorption of an alkaline agent and a reducing agent.

Another object of the present invention is to provide a solvent composition for one-step permanent wave and hair straightener, which does not damage hair nor irritate the scalp, and which does not emit an odor.

Still another object of the present invention is to provide a permanent wave agent and a hair straightening agent comprising the composition for one-step permanent wave and hair straightener.

In a first embodiment of the invention there is provided a solvent composition for one-step permanent wave and hair straightener, comprising: 0.01-10 wt % of amino acid or a derivative thereof; 0.01-10 wt % of protein or a derivative thereof; 0.1-10 wt % of a reducing agent; 0.1-10 wt % of an alkaline agent; 0.01-20 wt % of an amino acid-based surfactant; 0.01-5 wt % of organic acid; 0.01-10 wt % of alcohol; 0.01-10 wt % of urea; and water (balance).

In particular embodiments, the solvent composition may further comprise 0.1-30 parts by weight of at least one fluorine compound based on 100 parts by weight of the total amount of the solvent composition. In yet more particular embodiments, the at least one fluorine compound may be selected from the group consisting of methyl perfluorobutyl ether, methyl perfluoroisobutyl ether, ethyl perfluorobutyl ethyl, and ethyl perfluoroisobutyl ether.

In additional embodiments, the amino acid and the derivative thereof may be one or more selected from the group consisting of histidine, lysine, arginine, glutamic acid, tricine, and derivatives thereof.

In other embodiments, the protein and the derivative thereof may be one or more selected from the group consisting of keratin protein, soy protein, silk protein, collagen protein, egg-white protein, milk protein, wool protein, wheat protein, and derivatives thereof.

In particular embodiments, the reducing agent may be at least one selected from the group consisting of thioglycolic acid, thioglycollate monoglycerol ester, thiolactic acid, cysteine, cysteamine, N-acetyl-L-cysteine, thioacetic acid, and salts thereof.

In yet other particular embodiments, the solvent composition is comprised of at least one alkaline agent selected from the group consisting of ethanolamine, sodium hydroxide, potassium hydroxide, potassium carbonate, and sodium carbonate.

In specific embodiments, the amino acid-based surfactant may be at least one selected from the group consisting of sodium cocoyl glutamate, potassium cocoyl glycinate, sodium myristoyl glutamate, sodium lauroyl sarcosinate, sodium palmitoyl sarcosinate, sodium lauroyl aspartate, and sodium methylcocoyl taurate.

In additional embodiments, the organic acid may be at least one selected from the group consisting of lactic acid, citric acid, tartaric acid, glycolic acid, malic acid, and succinic acid.

In yet other embodiments, the amino acid-based surfactant has a hydrophilic amino acid group selected from the group consisting of glutamate, glycinate, sarcosinate, aspartate, and taurate.

Another embodiment of the present invention provides a permanent wave agent and a hair straightening agent, comprising the solvent composition.

### DETAILED DESCRIPTION OF SPECIFIC EMBODIMENTS

It has been found that the efficiency of curling and straightening hair and the ability to maintain the curled and straightened hair can be improved when a solvent composition for one-step permanent wave and hair straightener includes: a specific amino acid-based surfactant which has high affinity for hair components (protein and amino acid), which induces a rapid cystine reduction reaction, and which can improve the penetration and action of a reducing agent by decreasing the repellency between the amino acid-based surfactant and hair protein; organic acid accelerating the cutting and natural oxidizing of cystine bonds; and urea accelerating the absorption of an alkaline agent and a reducing agent.

Embodiments of the present invention provides a novel solvent composition for one-step permanent wave and hair straightener, which does not damage hair nor irritate the scalp, which does not emit an odor and which can greatly improve the efficiency of curling and straightening hair and the ability to maintain the curled and straightened hair.

In one embodiment, a one-step permanent wave and hair straightener includes: 0.01-10 wt % of amino acid or a derivative thereof; 0.01-10 wt % of protein or a derivative thereof; 0.1-10 wt % of a reducing agent; 0.1-10 wt % of an alkaline agent; 0.01-20 wt % of an amino acid-based surfactant; 0.01-5 wt % of organic acid; 0.01-10 wt % of alcohol; 0.01-10 wt % of urea; and water (balance).

In this case, the amount of water is suitably adjusted such that the total amount of the solvent composition can be maintained at 100 wt %. That is, the solvent composition includes water as a balance such that the total amount of the solvent composition including the components and the water can be adjusted to 100 wt %.

The solvent compositions for one-step permanent wave and hair straightener according to embodiments of the invention are facilitated by the sulfur bond attributable to the dehydration and natural oxidation of cystine.

Hydrolysis: R—S—S—R+H₂O-------->R—S—H+HO—S—R

Dehydration: R—S—H+HO—S—R------->R—S—S—R+H₂O

Reduction: R—S—S—R+H₂------->R—S—H+H—S—R

Natural oxidation: R—S—H+H—S—R+O₂------->R—S—S—R+H₂O

Since such reactions occur simultaneously, the reaction rate of the solvent composition becomes high, hair is rapidly curled and straightened, and the resilience of hair is easily maintained.

In the solvent compositions according to embodiments of the present invention, the amino acid and derivatives thereof and protein and derivatives thereof serve to protect hair. Concretely, the amino acid and the derivative thereof may be one or more selected from the group consisting of histidine, lysine, arginine, glutamic acid, tricine, and derivatives thereof. The protein and the derivative thereof may be one or more selected from the group consisting of keratin protein, soy protein, silk protein, collagen protein, egg-white protein, milk protein, wool protein, wheat protein, and derivatives thereof, and, preferably, may be selected from keratin protein, silk protein, and derivatives thereof.

Here, each of the amount of the amino acid and the derivative thereof and the amount of the protein and the derivative thereof is 0.01-10 wt %, preferably 0.5-10 wt %, and more preferably 0.8-5 wt %. An amount less than 0.01 wt % is insufficient to protect hair. Further, an amount greater than 10 wt % can inhibit the action of reducing agents.

In the solvent compositions according to embodiments of the present invention, a reducing agent serves to cleave disulfide bonds. There may be at least one reducing agent selected from the group consisting of thioglycolic acid, thioglycollate monoglycerol ester, thiolactic acid, cysteine, cysteamine, N-acetyl-L-cysteine, thioacetic acid, and salts thereof.

The amount of the reducing agent is 0.1-10 wt %, preferably 0.5-8 wt %, and more preferably 3-7 wt %. A reducing agent concentration less than 0.1 wt % does not suffice for purposes of the present invention. Further, if the amount of reducing agent is more than 10 wt %, hair is damaged.

In solvent compositions according to embodiments of the present invention, an alkaline agent serves to expand the cuticle of hair and accelerate the activity of a reducing agent. There may be at least one alkaline agent selected from the group consisting of strong aqueous ammonia, ethanolamine, sodium hydroxide, potassium hydroxide, potassium carbonate, and sodium carbonate. Various combinations of alkaline agents may also be used.

The amount of the alkaline agent is 0.1-10 wt %, preferably 0.5-8 wt %, and more preferably 2.0-5.0 wt %. Concentrations of alkaline agent less than 0.1 wt % do not sufficiently swell the hair (that is, the cuticle of hair does not sufficiently expand), with the result that the solvent composition does not sufficiently penetrate hair. Further, when the amount of the alkaline agent is more than 10 wt %, hair is damaged.

In solvent compositions according to embodiments of the present invention, an amino acid-based surfactant serves to enhance the penetration of the solvent composition into hair. In particular, since the amino acid-based surfactant has high affinity for protein and amino acid constituting hair, it can improve the penetration of a hydrophilic solvent composition, and particularly a reducing agent, into hair.

Further, the amino acid-based surfactant exhibits both cationic and anionic effects even in an acidic state in which organic acid exists, and thus improves both the adsorption of the solvent composition on hair by cations and the penetration of the solvent composition into hair. In particular, since taurate contains sulfur, it improves the affinity of the solvent composition for hair.

The amino acid-based surfactant includes a hydrophilic amino acid group and a hydrophobic aliphatic acid group, and reacts with alkali to form a salt. At least one hydrophilic amino acid group may be selected from the group consisting of glutamate, glycinate, sarcosinate, aspartate, and taurate.

Examples of the aliphatic acid group may include cocoylic acid, lauric acid, myristic acid, and stearic acid, preferably, cocoylic acid, lauric acid, and myristic acid. As the alkali, an alkaline agent such as an alkali metal salt, ethanolamine or the like may be used. In particular embodiments, sodium or potassium may be used as the alkaline metal.

In specific embodiments, the amino acid-based surfactant may be selected from the group consisting of sodium cocoyl glutamate, potassium cocoyl glycinate, sodium myristoyl glutamate, sodium lauroyl sarcosinate, sodium palmitoyl sarcosinate, sodium lauroyl aspartate, and sodium methylcocoyl taurate. Multiple amino acid-based surfactants and combinations thereof may also be used.

Among these amino acid-based surfactants, sodium cocoyl glutamate, potassium cocoyl glutamate, sodium myristoyl glutamate and sodium lauroyl aspartate are excellent in terms of softening power; potassium cocoyl glycinate, sodium lauroyl aspartate, sodium methylcocoyl taurate are excellent in terms of the softness of hair; and sodium lauroyl aspartate and sodium methylcocoyl taurate possess excellent straightening power. Specific and desirable effects can be achieved through particular combinations of amino acid-based surfactants.

The amount of the amino acid-based surfactant is 0.01-20 wt %, preferably 0.1-10 wt %, and more preferably 0.5-5 wt %. A concentration of amino acid-based surfactant less than 0.01 wt % is insufficient to serve as a surfactant. Further, when the amount of amino acid-based surfactant is more than 20 wt %, hair is damaged.

In solvent compositions according to embodiments of the present invention, organic acid serves to accelerate the natural oxidation of a reducing agent cleaving the cystine bond. The organic acid may be an organic acid of 10 or less carbon atoms. In specific embodiments, at least one organic acid may be selected from the group consisting of lactic acid, citric acid, tartaric acid, glycolic acid, malic acid, and succinic acid, and preferably may be selected from tartaric acid, lactic acid and glycolic acid.

The amount of the organic acid is 0.01-5 wt %, preferably 0.01-3 wt %, and more preferably 0.05-2.5 wt %. A concentration of organic acid less than 0.01 wt % cannot sufficiently accelerate natural oxidation. Further, when the amount of organic acid is more than 5 wt %, the organic acid influences the activity of a reducing agent and an alkaline agent, so that it is difficult to get a permanent wave and straighten hair.

In solvent compositions according to embodiments of the present invention, alcohol serves as a penetration-enhancing agent for enhancing the penetration of a reducing agent into hair.

The alcohol may be an alcohol of 10 or less carbon atoms. In specific embodiments, at least one alcohol is used, which may be selected from the group consisting of ethyl alcohol, benzyl alcohol, isopropyl alcohol, and phenylethyl alcohol.

The amount of the alcohol is 0.01-10 wt %, and preferably 0.1-8 wt %. An amount less than 0.01 wt % cannot sufficiently enhance the penetration of a reducing agent into hair. Further, when the amount of alcohol is more than 10 wt %, hair is damaged.

In embodiments of the present invention, urea serves as a penetrating agent for increasing the absorptivity of an alkaline agent such that a reducing agent can easily penetrate hair.

The amount of the urea is 0.01-10 wt %, preferably 0.1-8.0 wt %, and more preferably 0.1-5 wt %. An amount of the urea less than 0.01 wt % is insufficient, such that a reducing agent cannot easily penetrate hair. Further, when the amount of urea is more than 10 wt %, hair is damaged.

Meanwhile, in order to enhance oxidation, embodiments of the solvent composition of the present invention may further include a fluorine compound for loading oxidation gas.

The fluorine compound may be perfluorodecalin or the like. In specific embodiments, the fluorine compound may be selected from the group consisting of methyl perfluorobutyl ether, methyl perfluoroisobutyl ether, ethyl perfluorobutyl ethyl, and ethyl perfluoroisobutyl ether. Particular embodiments of the invention comprise at least one fluorine compound, and may comprise multiple fluorine compounds and combinations thereof.

The amount of the fluorine compound may be 0.1-30 parts by weight, preferably 5-20 parts by weight, and more preferably 10-15 parts by weight, based on 100 parts by weight of the total amount of the solvent composition. If the amount of the fluorine compound is less than 0.1 parts by weight, a small amount of oxygen is loaded, so that oxidation is not sufficiently enhanced. Further, if the amount of fluorine compound is more than 30 parts by weight, it is difficult to maintain the stability of the solvent composition, so that it is difficult to make the solvent composition into a product.

In addition to the above components, the solvent composition may further include various additives in order to provide functionality. Examples of the additives may include an antiseptic agent, an ultraviolet absorber, an antioxidant, an antibacterial agent, perfume, and the like.

Embodiments of the present invention further include a permanent wave agent and a hair straightening agent, comprising the solvent composition. The permanent wave agent and the hair straightening agent may be manufactured in the form of a solution, emulsion, lotion, cream, gel, aerosol or the like. Here, since the components used to maintain the form are identical with conventional forms, they are not particularly limited.

Hereinafter, particular embodiments of the present invention will be described in more detail with reference to the following Examples. However, the scope of the present invention is not limited thereto.

### Example 1

Solvent compositions for one-step permanent wave and hair straightener were prepared by mixing the components given in Table 1 below in the predetermined amounts given in Table 1 below.

TABLE 1

| Components (g) | Example 1-1 | Example 1-2 | Example 1-3 |
|---|---|---|---|
| Amino acid and | arginine | arginine | arginine |
| derivative thereof | (0.2) | (0.2) | (0.2) |
| Amino acid- | disodium cocoyl glutamate | disodium cocoyl glutamate | disodium cocoyl glutamate |
| based surfactant | (1.0) | (1.0) | (1.0) |
| Protein and | keratin protein (1.0) | keratin protein (1.0) | keratin protein (1.0) |
| derivative thereof | | | |
| Reducing agent | cysteamine hydrochloride | cysteamine hydrochloride | cysteamine hydrochloride |
| | (2.0) | (2.0) | (2.0) |
| | cysteine HCl (1.5) | cysteine HCl (1.5) | cysteine HCl (1.5) |
| | ethanolamine thioglycolate | ethanolamine thioglycolate | ethanolamine thioglycolate |
| | (50%) (14) | (50%) (14) | (50%) (14) |
| Organic acid | citric acid (0.5) | citric acid (0.5) | citric acid (0.5) |
| Urea | urea (5.0) | urea (5.0) | urea (5.0) |
| Alcohol | ethanol (10) | ethanol (10) | ethanol (10) |
| Fluorine | — | methyl perfluorobutyl ether | ethyl perfluorobutyl ether |
| compound | | (1.0) | (1.0) |
| Aqueous | aqueous ammonia (28%) | aqueous ammonia (28%) | aqueous ammonia (28%) |
| ammonia | (1.0) | (1.0) | (1.0) |
| (28%) | | | |
| chelating agent | disodium | disodium | disodium |
| | ethylenediaminetetra- | ethylenediaminetetra- | ethylenediaminetetra- |
| | acetate | acetate | acetate |
| | (EDTA) | (EDTA) | (EDTA) |
| | (0.05) | (0.05) | (0.05) |
| Alkaline agent | monoethanolamine (4.0) | monoethanolamine (4.0) | monoethanolamine (4.0) |
| Purified water | (59.75) | (58.75) | (58.75) |

### Example 2

### Changing the Kind of an Amino Acid-Based Surfactant

Solvent compositions for one-step permanent wave and hair straightener were prepared in the same manner as Example 1, except that the kind of an amino acid-based surfactant was changed as given in Table 2 below.

TABLE 2

| Components (g) | Example 2-1 | Example 2-2 | Example 2-3 | Example 2-4 |
|---|---|---|---|---|
| Amino acid and | arginine | arginine | arginine | arginine |
| derivative thereof | (0.2) | (0.2) | (0.2) | (0.2) |
| Amino acid- | sodium cocoyl | sodium lauroyl | potassium cocoyl | sodium methylcocoyl |
| based surfactant | glutamate (1.0) | aspartate (1.0) | glycinate (1.0) | taurate (1.0) |
| Protein and | keratin protein (1.0) | keratin protein (1.0) | keratin protein (1.0) | keratin protein (1.0) |
| derivative thereof | | | | |
| Reducing agent | cysteamine | cysteamine | cysteamine | cysteamine |
| | hydrochloride | hydrochloride | hydrochloride | hydrochloride |
| | (2.0) | (2.0) | (2.0) | (2.0) |
| | cysteine HCl (1.5) | cysteine HCl (1.5) | cysteine HCl (1.5) | cysteine HCl (1.5) |
| | ethanolamine | ethanolamine | ethanolamine | ethanolamine |
| | thioglycolate (50%) | thioglycolate (50%) | thioglycolate (50%) | thioglycolate (50%) |
| | (14) | (14) | (14) | (14) |
| Organic acid | citric acid (0.5) | citric acid (0.5) | citric acid (0.5) | citric acid (0.5) |
| Urea | urea (5.0) | urea (5.0) | urea (5.0) | urea (5.0) |
| Alcohol | ethanol (10) | ethanol (10) | ethanol (10) | ethanol (10) |
| Aqueous | aqueous ammonia | aqueous ammonia | aqueous ammonia | aqueous ammonia |
| ammonia | (28%) (1.0) | (28%) (1.0) | (28%) (1.0) | (28%) (1.0) |
| (28%) | | | | |
| chelating agent | disodium | disodium | disodium | disodium |
| | ethylenediaminetetra- | ethylenediaminetetra- | ethylenediaminetetra- | ethylenediaminetetra- |
| | acetate (EDTA) | acetate (EDTA) | acetate (EDTA) | acetate (EDTA) |
| | (0.05) | (0.05) | (0.05) | (0.05) |
| Alkaline agent | monoethanolamine | monoethanolamine | monoethanolamine | monoethanolamine |
| | (4.0) | (4.0) | (4.0) | (4.0) |
| Purified water | (59.75) | (59.75) | (59.75) | (59.75) |

### Example 3

### Changing the Kind of Organic Acid

Solvent compositions for one-step permanent wave and hair straightener were prepared in the same manner as Example 1, except that the kind of organic acid was changed as given in Table 3 below.

TABLE 3

| Components (g) | Example 3-1 | Example 3-2 | Example 3-3 | Example 3-4 |
|---|---|---|---|---|
| Amino acid and | arginine | arginine | arginine | arginine |
| derivative thereof | (0.2) | (0.2) | (0.2) | (0.2) |
| Amino acid- | disodium cocoyl | disodium cocoyl | disodium cocoyl | disodium cocoyl |
| based surfactant | glutamate (1.0) | glutamate (1.0) | glutamate (1.0) | glutamate (1.0) |
| Protein and | keratin protein (1.0) | keratin protein (1.0) | keratin protein (1.0) | keratin protein (1.0) |
| derivative thereof | | | | |
| Reducing agent | cysteamine | cysteamine | cysteamine | cysteamine |
| | hydrochloride | hydrochloride | hydrochloride | hydrochloride |
| | (2.0) | (2.0) | (2.0) | (2.0) |
| | cysteine HCl (1.5) | cysteine HCl (1.5) | cysteine HCl (1.5) | cysteine HCl (1.5) |
| | ethanolamine | ethanolamine | ethanolamine | ethanolamine |
| | thioglycolate (50%) | thioglycolate (50%) | thioglycolate (50%) | thioglycolate (50%) |
| | (14) | (14) | (14) | (14) |
| Organic acid | lactic acid (0.5) | glycolic acid (0.5) | citric acid (0.5) | tartaric acid (0.5) |
| Urea | urea (5.0) | urea (5.0) | urea (5.0) | urea (5.0) |
| Alcohol | ethanol (10) | ethanol (10) | ethanol (10) | ethanol (10) |
| Aqueous | aqueous ammonia | aqueous ammonia | aqueous ammonia | aqueous ammonia |
| ammonia | (28%) (1.0) | (28%) (1.0) | (28%) (1.0) | (28%) (1.0) |
| (28%) | | | | |
| chelating agent | disodium | disodium | disodium | disodium |
| | ethylenediaminetetra- | ethylenediaminetetra- | ethylenediaminetetra- | ethylenediaminetetra- |
| | acetate (EDTA) | acetate (EDTA) | acetate (EDTA) | acetate (EDTA) |
| | (0.05) | (0.05) | (0.05) | (0.05) |
| Alkaline agent | monoethanolamine | monoethanolamine | monoethanolamine | monoethanolamine |
| | (4.0) | (4.0) | (4.0) | (4.0) |
| Purified water | (59.75) | (59.75) | (59.75) | (59.75) |

### Example 4

Solvent compositions for one-step permanent wave and hair straightener were prepared in the same manner as Example 1, except that the components and contents thereof were changed as given in Table 4 below.

TABLE 4

| Compo- | | | | | | | | |
|---|---|---|---|---|---|---|---|---|
| nents (g) | Exp. 4-1 | Exp. 4-2 | Exp. 4-3 | Exp. 4-4 | Exp. 4-5 | Exp. 4-6 | Exp. 4-7 | Exp. 4-8 |
| Amino acid | arginine | arginine | arginine | arginine | arginine | arginine | arginine | arginine |
| and | (0.2) | (0.2) | (0.2) | (0.2) | (0.2) | (0.2) | (0.2) | (0.2) |
| derivative | | | | | | | | |
| thereof | | | | | | | | |
| Amino acid- | sodium | sodium | sodium | sodium | sodium | sodium | sodium | sodium |
| based | lauroyl | lauroyl | lauroyl | lauroyl | lauroyl | lauroyl | lauroyl | lauroyl |
| surfactant | aspartate | aspartate | aspartate | aspartate | aspartate | aspartate | aspartate | aspartate |
| | (3.0) | (3.0) | (3.0) | (3.0) | (3.0) | (3.0) | (3.0) | (3.0) |
| Protein and | keratin | keratin | keratin | keratin | keratin | keratin | keratin | keratin |
| derivative | protein | protein | protein | protein | protein | protein | protein | protein |
| thereof | (1.0) | (1.0) | (1.0) | (1.0) | (1.0) | (1.0) | (1.0) | (1.0) |
| Reducing | cysteamine | cysteamine | cysteamine | cysteamine | cysteamine | cysteamine | cysteamine | cysteamine |
| agent | hydro- | hydro- | hydro- | hydro- | hydro- | hydro- | hydro- | hydro- |
| | chloride | chloride | chloride | chloride | chloride | chloride | chloride | chloride |
| | (2.0) | (2.0) | (2.0) | (2.0) | (2.0) | (2.0) | (2.0) | (2.0) |
| | cysteine HCl | cysteine HCl | cysteine HCl | cysteine HCl | cysteine HCl | cysteine HCl | cysteine HCl | cysteine HCl |
| | (1.5) | (1.5) | (1.5) | (1.5) | (1.5) | (1.5) | (1.5) | (1.5) |
| | ethanolamine | ethanolamine | ethanolamine | ethanolamine | ethanolamine | ethanolamine | ethanolamine | ethanolamine |
| | thioglycolate | thioglycolate | thioglycolate | thioglycolate | thioglycolate | thioglycolate | thioglycolate | thioglycolate |
| | (50%) (14) | (50%) (14) | (50%) (14) | (50%) (14) | (50%) (14) | (50%) (14) | (50%) (14) | (50%) (14) |
| Organic acid | lactic acid | lactic acid | lactic acid | lactic acid | lactic acid | lactic acid | lactic acid | lactic acid |
| | (0.5) | (0.5) | (0.5) | (0.1) | (1.0) | (1.0) | (1.0) | (1.0) |
| Urea | urea | urea | urea | urea | urea | urea | urea | urea |
| | (1.0) | (5.0) | (10) | (5.0) | (5.0) | (5.0) | (5.0) | (5.0) |
| Alcohol | ethanol | ethanol | ethanol | ethanol | ethanol | ethanol | ethanol | ethanol |
| | (10) | (10) | (10) | (10) | (10) | (10) | (10) | (10) |
| Aqueous | aqueous | aqueous | aqueous | aqueous | aqueous | aqueous | aqueous | aqueous |
| ammonia | ammonia | ammonia | ammonia | ammonia | ammonia | ammonia | ammonia | ammonia |
| (28%) | (28%) (1.0) | (28%) (1.0) | (28%) (1.0) | (28%) (1.0) | (28%) (1.0) | (28%) (1.0) | (28%) (1.0) | (28%) (1.0) |
| chelating | disodium | disodium | disodium | disodium | disodium | disodium | disodium | disodium |
| agent | ethylene- | ethylene- | ethylene- | ethylene- | ethylene- | ethylene- | ethylene- | ethylene- |
| | diamine- | diamine- | diamine- | diamine- | diamine- | diamine- | diamine- | diamine- |
| | tetraacetate | tetraacetate | tetraacetate | tetraacetate | tetraacetate | tetraacetate | tetraacetate | tetraacetate |
| | (EDTA) | (EDTA) | (EDTA) | (EDTA) | (EDTA) | (EDTA) | (EDTA) | (EDTA) |
| | (0.05) | (0.05) | (0.05) | (0.05) | (0.05) | (0.05) | (0.05) | (0.05) |
| Alkaline | monoethanol- | monoethanol- | monoethanol- | monoethanol- | monoethanol- | monoethanol- | monoethanol- | monoethanol- |
| agent | amine | amine | amine | amine | amine | amine | amine | amine |
| | (4.0) | (4.0) | (4.0) | (4.0) | (4.0) | (4.0) | (4.0) | (4.0) |
| Purified | (61.75) | (57.75) | (52.75) | (58.65) | (57.25) | (53.25) | (60.25) | (55.75) |
| water | | | | | | | | |

### Comparative Examples 1 to 8

Solvent compositions for one-step permanent wave and hair straightener were prepared in the same manner as Example 1, except that the components and contents thereof were changed as given in Table 5 below.

TABLE 5

| Compo- | | | | | | | | |
|---|---|---|---|---|---|---|---|---|
| nents(g) | Comp. Ex. 1 | Comp. Ex. 2 | Comp. Ex. 3 | Comp. Ex. 4 | Comp. Ex. 5 | Comp. Ex. 6 | Comp. Ex. 7 | Comp. Ex. 8 |
| Amino acid | arginine | arginine | arginine | arginine | arginine | arginine | arginine | arginine |
| and | (0.2) | (0.2) | (0.2) | (0.2) | (0.2) | (0.2) | (0.2) | (0.2) |
| derivative | | | | | | | | |
| thereof | | | | | | | | |
| Surfactant | sodium lauroyl | sodium | — | — | ammonium | disodium | sodium lauryl | sodium lauryl |
| | aspartate | methylcocoyl | | | cocoyl | capryloyl | sulfate | sulfate |
| | (1.0) | taurate | | | sarcosinate | glutamate | (1.0) | (1.0) |
| | | (1.0) | | | (1.0) | (1.0) | | |
| Protein and | keratin | keratin | keratin | keratin | keratin | keratin | keratin | keratin |
| derivative | protein | protein | protein | protein | protein | protein | protein | protein |
| thereof | (1.0) | (1.0) | (1.0) | (1.0) | (1.0) | (1.0) | (1.0) | (1.0) |
| Reducing | cysteamine | cysteamine | cysteamine | cysteamine | cysteamine | cysteamine | cysteamine | cysteamine |
| agent | hydro- | hydro- | hydro- | hydro- | hydro- | hydro- | hydro- | hydro- |
| | chloride | chloride | chloride | chloride | chloride | chloride | chloride | chloride |
| | (2.0) | (2.0) | (2.0) | (2.0) | (2.0) | (2.0) | (2.0) | (2.0) |
| | cysteine HCl | cysteine HCl | cysteine HCl | cysteine HCl | cysteine HCl | cysteine HCl | cysteine HCl | cysteine HCl |
| | (1.5) | (1.5) | (1.5) | (1.5) | (1.5) | (1.5) | (1.5) | (1.5) |
| Organic acid | — | — | citric acid | tartaric acid | — | — | tartaric acid | tartaric acid |
| | | | (0.5) | (0.5) | | | (0.5) | (0.5) |
| Urea | urea | urea | urea | urea | urea | urea | urea | urea |
| | (5.0) | (5.0) | (5.0) | (5.0) | (5.0) | (5.0) | (5.0) | (5.0) |
| Alcohol | ethanol | ethanol | ethanol | ethanol | ethanol | ethanol | ethanol | ethanol |
| | (10) | (10) | (10) | (10) | (10) | (10) | (10) | (10) |
| Aqueous | aqueous | aqueous | aqueous | aqueous | aqueous | aqueous | aqueous | aqueous |
| ammonia | ammonia | ammonia | ammonia | ammonia | ammonia | ammonia | ammonia | ammonia |
| (28%) | (28%) (1.0) | (28%) (1.0) | (28%) (1.0) | (28%) (1.0) | (28%) (1.0) | (28%) (1.0) | (28%) (1.0) | (28%) (1.0) |
| chelating | disodium | disodium | disodium | disodium | disodium | disodium | disodium | disodium |
| agent | ethylene- | ethylene- | ethylene- | ethylene- | ethylene- | ethylene- | ethylene- | ethylene- |
| | diamine- | diamine- | diamine- | diamine- | diamine- | diamine- | diamine- | diamine- |
| | tetraacetate | tetraacetate | tetraacetate | tetraacetate | tetraacetate | tetraacetate | tetraacetate | tetraacetate |
| | (EDTA) | (EDTA) | (EDTA) | (EDTA) | (EDTA) | (EDTA) | (EDTA) | (EDTA) |
| | (0.05) | (0.05) | (0.05) | (0.05) | (0.05) | (0.05) | (0.05) | (0.05) |
| Alkaline | ethanolamine | ethanolamine | ethanolamine | ethanolamine | ethanolamine | ethanolamine | ethanolamine | ethanolamine |
| agent | thioglycolate | thioglycolate | thioglycolate | thioglycolate | thioglycolate | thioglycolate | thioglycolate | thioglycolate |
| | (50%) (14) | (50%) (14) | (50%) (14) | (50%) (14) | (50%) (14) | (50%) (14) | (50%) (14) | (50%) (14) |
| | monoethanol- | monoethanol- | monoethanol- | monoethanol- | monoethanol- | monoethanol- | monoethanol- | monoethanol- |
| | amine | amine | amine | amine | amine | amine | amine | amine |
| | (4.0) | (4.0) | (4.0) | (4.0) | (4.0) | (4.0) | (4.0) | (4.0) |
| Purified | (59.75) | (59.75) | (59.75) | (59.75) | (59.75) | (59.75) | (59.75) | (59.75) |
| water | | | | | | | | |

### Comparative Examples 9 and 10

### Conventional Solvent Compositions for One-Step Permanent Wave and Hair Straightener

Solvent compositions for one-step permanent wave and hair straightener were prepared in the same manner as Example 1, except that the components and contents thereof were changed as given in Table 6 below.

TABLE 6

| | Comparative | Comparative |
|---|---|---|
| Components (g) | Example 9 | Example 10 |
| Ethanolamine | 16 | 16 |
| thioglycolate (50%) | | |
| Potassium cyanide | 0.6 | 0.6 |
| Potassium thiocyanide | 1.2 | 1.2 |
| Trihydroxymethyl phosphine | — | 1.2 |
| Sodium form aldehyde | — | 0.5 |
| sulphoxylate | | |
| Diethanolamine | 8.0 | 8.0 |
| Ethyl alcohol | 7.0 | 7.0 |
| Sodium carbonate | 1.0 | 1.0 |
| HPMC #40,000 | 2.0 | 2.0 |
| Disodium ethylene- | 0.4 | 0.4 |
| diaminetetraacetate | | |
| Purified water | 36.2 | 37.9 |

### Comparative Example 11

A solvent composition for one-step permanent wave and hair straightener was prepared using a first reductive solution and a second oxidative solution, each including the components and contents given in Table 7 below.

TABLE 7

| First reductive | Content | Second oxidative | Content |
|---|---|---|---|
| solution | (g) | solution | (g) |
| Hydroxyethyl | 0.5 | Glycerin | 1 |
| cellulose | | | |
| Polyquarternium-10 | 0.5 | Hydroxyethyl | 0.2 |
| | | cellulose | |
| Carbomer | 0.5 | Polyquarternium-10 | 0.2 |
| Glycerin | 1.0 | Carbomer | 0.3 |
| Etidronic acid | 0.1 | Sodium carboxymethyl | 0.2 |
| | | cellulose | |
| Propylene glycol | 2.0 | Propylene glycol | 2.0 |
| L-cysteine | 7.0 | Etidronic acid | 0.1 |
| hydrochloride | | | |
| Thioglycolic | 2.0 | Phosphoric acid | 0.05 |
| acid (50%) | | | |
| Urea | 1.0 | Sodium phosphate | 0.2 |
| Monoethanolamine | 5.0 | Polysorbate 80 | 1.0 |
| Polysorbate 20 | 1.0 | Perfume | 0.1 |
| Perfume | 0.1 | Hydrogen peroxide | 6.00 |
| | | water (35%) | |
| Purified water | 79.3 | Purified water | 88.65 |

### Test Examples

The physical properties of the solvent compositions for permanent wave and hair straightener of Examples 1 to 4 and Comparative Examples 1 to 11 were measured as follows, and the results thereof are given in Table 8 below.

### Test Example 1

### Wave Formation Rate Test

The wave formation rates of hair were measured as follows using the solvent compositions for permanent wave and hair straightener of Examples 1 to 4 and Comparative Examples 1 to 11.

The wave formation rates were measured by a wave formation tester using the Kirby method as follows. One side of a bundle of hair was fixed on a plastic tool, and the other side thereof was fixed on a pin rod by hanging it on the pin rod in a zigzag. The bundle of hair fixed on the pin rod was put into a vessel filled with each of the solvent compositions for permanent wave and hair straightener of Examples 1 to 4 and Comparative Examples 1 to 11, and was then left in a thermostatic bath of 25° C. for 20 minutes. Subsequently, the bundle of hair was washed with water, unfixed and then dried to form a wave. Thereafter, the waved hair was naturally disposed on a glass plate provided with graduations, and the wave formation rate thereof was calculated by the following Equation 1:

Wave formation rate(%)=100−{100×(b−a)/(c−a)} Equation 1

wherein a is the distance between the first pin rod and the sixth pin rod of the plastic tool, b is the distance between five curved points of the waved hair, and c is the distance between the five curved points of the waved hair when it is straightened.

### Test Example 2

### Wave Maintenance Rate Test

A bundle of the waved hair obtained from the Test Example 1 was respectively put into a test tube filled with a 30% sodium lauryl sulfate solution (pH=8.5) and a test tube filled with a sweat solution, and was then left in a thermostatic bath of 45° C. for 1 hour. Subsequently, the bundle of waved hair was taken out from the test tube, washed with water, dried, and then naturally disposed on a glass plate provided with graduations. Thereafter, the wave rate of the waved hair after the wave maintenance test was calculated, so that the wave maintenance rate thereof could be calculated by the following Equation 2. In this case, the sweat solution includes 0.5 g of L-cysteine hydrochloride (monohydrate), 0.5 g of sodium chloride and 5.0 g of disodium phosphate (dodecahydrate), and its pH was set to 110.0 using sodium hydroxide, and its volume was set to 1 L using purified water.

Wave maintenance rate(%)=(wave rate after wave maintenance test/wave rate before wave maintenance test)×100 Equation 2

### Test Example 3

### Hair Straightening Test

Hair was washed and dried, and then each of the solvent compositions for permanent wave and hair straightener of Examples 1 to 4 and Comparative Examples 1 to 11 was applied to the hair. Subsequently, the hair was fixed on a rod for hair straightening, and then each of the solvent compositions of Examples 1 to 4 and Comparative Examples 1 to 11 was further applied thereto. Thereafter, the hair was covered with a vinyl cover, heat-treated by an electric cap for 15 minutes, naturally left for 30 minutes in a state in which the electric cap was removed. Then, the vinyl cover was removed from the hair, and then the hair was further left for 15 minutes. Subsequently, the hair was unfixed from the rod, and was then sufficiently washed with water. Thereafter, twenty hair beauty experts observed the state of the hair, and then evaluated the straightening ability of each of the solvent compositions. For the evaluation criterion, the highest point was set to 10 (good), and the lowest point was set to 1 (poor).

### Test Example 4

### Hair Damage Test

Discolored hair, dyed hair and normal hair having a length of 15-20 cm were shampooed and then dried with a towel. Each of the solvent compositions for permanent wave and hair straightener of Examples 1 to 4 and Comparative Examples 1 to 11 was uniformly applied to the towel-dried hair, and then this hair was wound on a rod and then left for 20 minutes. Subsequently, the hair was unwound from the rod, washed with flowing water and then dried. Thereafter, ten hair beauty experts observed the treated hair and the non-treated hair using a microscope, and then evaluated the degree of damage of the cuticle layer of the hair. For the evaluation criterion, the highest point was set to 10 (good), and the lowest point was set to 1 (poor).

### Test Example 5

### Measurement of Odor after Applying Solvent Composition

The odor of the solvent compositions applied to hair was measured using ten samples. That is, the hairs of women aged tens, twenties, thirties, forties and fifties were divided into two equal parts, and then each of the solvent compositions for permanent wave and hair straightener of Examples 1 to 4 and Comparative Examples 1 to 11 were respectively applied to the bisected hairs, and then the odors thereof were measured by hair beauty experts. For the evaluation criterion, the highest point was set to 10 (good), and the lowest point was set to 1 (poor).

### Test Example 6

### Scalp Irritation Test

The skin, which is disposed behind the ear and around which hair is grown, was cleanly washed with soap and water, and then each of the solvent compositions for permanent wave and hair straightener of Examples 1 to 4 and Comparative Examples 1 to 11 was applied onto the skin in the size of a coin. Subsequently, the skin was left for 20-30 minutes or 48 hours, and then washed with water. Then, red spots, black spots, swelling, itching and the like of the skin were observed. As test objects, ten men aged tens to fifties and ten women aged tens to fifties were randomly selected, and the evaluation results thereof were averaged. For the evaluation criterion, the highest point was set to 10 (good), and the lowest point was set to 1 (poor).

### Test Example 7

### Hair Discoloration Test

Hair was washed and dried, and then each of the solvent compositions for permanent wave and hair straightener of Examples 1 to 4 and Comparative Examples 1 to 11 was applied to the hair. Subsequently, the hair was wound on a rod, and then covered with a vinyl cover. Thereafter, the hair was heat-treated by an electric cap for 15 minutes, and then naturally left for 30 minutes in a state in which the electric cap was removed. Then, the vinyl cover was removed from the hair, and then the hair was further left for 15 minutes. Subsequently, the hair was unwound from the rod, and then sufficiently washed with water. Thereafter, ten hair beauty experts compared the treat hair with non-treated hair, and then evaluated the degree of hair discoloration. For the evaluation criterion, the highest point was set to 10 (good), and the lowest point was set to 1 (poor).

TABLE 8

| | Wave | Wave | Hair | | | | Hair |
|---|---|---|---|---|---|---|---|
| | forma- | mainte- | straight- | Damage | | Scalp | dis- |
| | tion | nance | ening | of | | irrita- | color- |
| Class. | rate (%) | rate (%) | effect | hair | Odor | tion | ation |
| Example 1-1 | 50.3 | 77.1 | 7 | 7 | 8 | 8 | 8 |
| Example 1-2 | 50.2 | 80.5 | 8 | 7 | 7 | 8 | 8 |
| Example 1-3 | 50.8 | 80.5 | 8 | 7 | 7 | 8 | 8 |
| Example 2-1 | 53.3 | 78.6 | 7 | 7 | 7 | 8 | 8 |
| Example 2-2 | 52.2 | 82.8 | 8 | 8 | 8 | 9 | 8 |
| Example 2-3 | 50.6 | 80.0 | 7 | 8 | 8 | 8 | 8 |
| Example 2-4 | 54.3 | 84.6 | 8 | 8 | 8 | 9 | 8 |
| Example 3-1 | 51.1 | 78.4 | 7 | 7 | 7 | 8 | 8 |
| Example 3-2 | 53.1 | 80.0 | 7 | 7 | 7 | 8 | 9 |
| Example 3-3 | 54.6 | 83.3 | 8 | 7 | 8 | 9 | 9 |
| Example 3-4 | 55.6 | 85.6 | 9 | 8 | 8 | 9 | 9 |
| Example 4-1 | 50.6 | 79.2 | 6 | 8 | 8 | 8 | 9 |
| Example 4-2 | 50.5 | 84.0 | 7 | 8 | 8 | 8 | 8 |
| Example 4-3 | 53.2 | 82.0 | 8 | 7 | 8 | 8 | 8 |
| Example 4-4 | 51.6 | 82.6 | 7 | 7 | 7 | 8 | 8 |
| Example 4-5 | 50.1 | 84.5 | 7 | 7 | 7 | 8 | 8 |
| Example 4-6 | 51.1 | 83.0 | 7 | 7 | 7 | 8 | 8 |
| Example 4-7 | 53.6 | 81.1 | 8 | 8 | 8 | 8 | 9 |
| Example 4-8 | 52.3 | 82.6 | 8 | 7 | 7 | 8 | 8 |
| Comparative | 45.9 | 75.7 | 7 | 6 | 6 | 7 | 7 |
| Example 1 | | | | | | | |
| Comparative | 46.3 | 77.4 | 7 | 7 | 7 | 7 | 7 |
| Example 2 | | | | | | | |
| Comparative | 47.6 | 78.5 | 7 | 6 | 7 | 7 | 7 |
| Example 3 | | | | | | | |
| Comparative | 47.1 | 78.7 | 7 | 6 | 7 | 7 | 7 |
| Example 4 | | | | | | | |
| Comparative | 42.1 | 77.8 | 6 | 6 | 6 | 6 | 7 |
| Example 5 | | | | | | | |
| Comparative | 43.2 | 76.2 | 7 | 6 | 6 | 6 | 7 |
| Example 6 | | | | | | | |
| Comparative | 48.9 | 74.4 | 7 | 6 | 7 | 6 | 7 |
| Example 7 | | | | | | | |
| Comparative | 49.2 | 74.6 | 7 | 6 | 7 | 6 | 6 |
| Example 8 | | | | | | | |
| Comparative | 41.6 | 72.3 | 5 | 6 | 5 | 6 | 6 |
| Example 9 | | | | | | | |
| Comparative | 43.1 | 71.9 | 6 | 6 | 6 | 6 | 7 |
| Example 10 | | | | | | | |
| Comparative | 49.3 | 82.5 | 8 | 5 | 5 | 5 | 5 |
| Example 11 | | | | | | | |

As shown in Table 8 above, it can be presumed that the wave formation rates, wave maintenance rates and hair straightening effects of the solvent compositions of Examples 1 to 4 are remarkably higher than those of the solvent compositions of Comparative Examples 1 to 11, and that the solvent compositions of Examples 1 to 4 are equal to superior to the solvent compositions of Comparative Examples 1 to 11 in terms of hair damage, smell emission, scalp irritation and hair discoloration.

As described above, the solvent composition for one-step permanent wave and hair straightener according to embodiments of the present invention is advantageous in that the efficiency for curling hair becomes high because the time it takes to curl hair is shortened, and in that the ability to maintain the curled and straightened hair is excellent.

Further, the solvent composition according to embodiments of the present invention is advantageous in that it does not damage hair, does not irritate the scalp, does not emit an odor, and does not discolor hair.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A solvent composition for one-step permanent wave and hair straightener, comprising:
0.01-10 wt % of amino acid or a derivative thereof;
0.01-10 wt % of protein or a derivative thereof;
0.1-10 wt % of a reducing agent;
0.1-10 wt % of an alkaline agent;
0.01-20 wt % of an amino acid-based surfactant;
0.01-5 wt % of organic acid;
0.01-10 wt % of alcohol;
0.01-10 wt % of urea; and
water (balance).

2. The solvent composition according to claim 1, further comprising: 0.1-30 parts by weight of at least one fluorine compound based on 100 parts by weight of the total amount of the solvent composition.

3. The solvent composition according to claim 2, wherein at least one fluorine compound is selected from the group consisting of methyl perfluorobutyl ether, methyl perfluoroisobutyl ether, ethyl perfluorobutyl ethyl, and ethyl perfluoroisobutyl ether.

4. The solvent composition according to claim 1, wherein the amino acid and the derivative thereof are one or more selected from the group consisting of histidine, lysine, arginine, glutamic acid, tricine, and derivatives thereof.

5. The solvent composition according to claim 1, wherein the protein and the derivative thereof are one or more selected from the group consisting of keratin protein, soy protein, silk protein, collagen protein, egg-white protein, milk protein, wool protein, wheat protein, and derivatives thereof.

6. The solvent composition according to claim 1, wherein the reducing agent is at least one selected from the group consisting of thioglycolic acid, thioglycollate monoglycerol ester, thiolactic acid, cysteine, cysteamine, N-acetyl-L-cysteine, thioacetic acid, and salts thereof.

7. The solvent composition according to claim 1, wherein the alkaline agent is at least one selected from the group consisting of ethanolamine, sodium hydroxide, potassium hydroxide, potassium carbonate, and sodium carbonate.

8. The solvent composition according to claim 1, wherein the amino acid-based surfactant is at least one selected from the group consisting of sodium cocoyl glutamate, potassium cocoyl glycinate, sodium myristoyl glutamate, sodium lauroyl sarcosinate, sodium palmitoyl sarcosinate, sodium lauroyl aspartate, and sodium methylcocoyl taurate.

9. The solvent composition according to claim 1, wherein the organic acid is at least one selected from the group consisting of lactic acid, citric acid, tartaric acid, glycolic acid, malic acid, and succinic acid.

10. A permanent wave agent and a hair straightening agent, comprising the solvent composition of any one of claims 1 to 9.

11. The solvent composition according to claim 1, wherein the amino acid-based surfactant has a hydrophilic amino acid group selected from the group consisting of glutamate, glycinate, sarcosinate, aspartate, and taurate.
